(19) United States
(12) Patent Application Publication
(10) Pub. No.: US 2026/0301884 A1
(43) Pub. Date: Oct. 1, 2026

(54) Learning High Order Structures from Molecular SMILES/SELFIES Strings with Transformers

(71) Applicant: International Business Machines Corporation, Armonk, NY (US)

(72) Inventors: Thanh Lam Hoang, Maynooth (IE); Raúl Fernández Díaz, Dublin (IE); Mykhaylo Zayats, Dublin (IE); Vanessa Lopez Garcia, Dublin (IE)

(21) Appl. No.: 19/096,262

(22) Filed: Mar. 31, 2025

(51) Int. Cl. G16C 20/70; G16C 20/90

(52) U.S. Cl. CPC G16C 20/70; G16C 20/90

(57) ABSTRACT

A computer-implemented method to improve the performance of transformers in learning molecular representations from SMILES/SELFIES strings is presented. More specifically, an illustrative embodiment includes transformers learning high-order interaction between important substructures of molecules, and thus enhancing the ability of those transformers to learn and predict drug-related properties from molecular representations. According to other illustrative embodiments, a computer system and computer program product for improving the performance of transformers in learning molecular representations from SMILES/SELFIES strings are provided.

### BACKGROUND

The present disclosure relates generally to methods, systems and computer program products to improve the performance of machine learning models.

Pharmaceutical research and development is a large and growing undertaking. Drug trials are an important part of this research and development. These are costly endeavors.

### SUMMARY

The present disclosure relates generally to methods, systems and computer program products to improve the performance of transformers in learning molecular representations from SMILES (Simplified Molecular Input Line Entry System) and/or SELFIES (SELF-referencing embedded strings) character strings. More specifically, embodiments of this disclosure relate to transformers learning high-order interaction between important substructures of molecules, and thus enhancing the ability of those transformers to learn and predict drug-related properties from molecular representations.

Embodiments can leverage subgraphs of larger contextual molecular graphs to enhance the learning of transformers. This enables learning high or higher order correlations between multi-atom molecular subgraphs extracted from molecules rather than merely correlation between atoms or bonds.

In more detail, molecular (sub)graphs can be represented as SMILES and/or SELFIES character strings, which are sequence-based representations of these graphs. Transformers are employed to learn molecular representations from SMILES/SELFIES strings by predicting randomly masked tokens, similar to their training process for natural language. They are the most effective and popular methods for this task. However, random token masking fails to consider the substructures of the molecular graphs, resulting in the loss of important substructural information during training. In this work, we propose methods to train transformers to learn high-order substructures in SMILES/SELFIES strings. We demonstrate that transformers pretrained with high-order representation learning, in conjunction with regular learning methods, achieve state-of-the-art results on the ADMET (Absorption, Distribution, Metabolism, Excretion, and Toxicity) benchmarks for drug property prediction.

In particular, embodiments can include selecting tokens to mask SMILES and/or SELFIES character strings based on substructures defined in the molecular graphs, wherein a substructure is defined as a subgraph in a two-dimensional (2D) molecular graph or a substructure with atoms are closed to each other in a three-dimensional (3D) molecular graph. Embodiment can include using transformers to predict the tokens regarding the masked substructures to learn high-order interaction between substructures of the molecules. Embodiments can include creating a concise representation of substructure based on random hash or pertained compacted representation vectors of substructures via contrastive learning or auto-encoder and a prediction objective that predict the hash vectors/compacted representation vectors of the substructures.

The above summary is not intended to describe each illustrated embodiment or every implementation of the present disclosure.

### BRIEF DESCRIPTION OF THE DRAWINGS

FIG. 1 is a block diagram of a computing environment in accordance with an illustrative embodiment;

FIG. 2 is a block diagram of an architecture in accordance with an illustrative embodiment;

FIGS. 3A-3B are diagrams of (A) a molecule with a SMILES representation of the molecule and (B) a substructure masked version of the representation processed by transformers to predict the masked substructures in accordance with an illustrative embodiment;

FIGS. 4A-4B are diagrams of (A) an example of combining substructure mask prediction with single ascii character mask prediction and a hash vector prediction from the former and (B) construction of a substructure hash vector from a molecular graph in accordance with an illustrative embodiment;

FIGS. 5A-5B are diagrams of (A) Elo ratings {overall SmilesGraph ranked 2nd in terms of Elo rating} and (B) Wilcox on Signed rank test results in an ablation study in accordance with an illustrative embodiment;

FIG. 6 is a flow chart of a process in accordance with an illustrative embodiment; and

FIG. 7 is a block diagram of a data processing system in accordance with an illustrative embodiment.

### DETAILED DESCRIPTION

Embodiments include a computer implemented method, comprising: encoding, using a set of processors, a molecular graph of a chemical molecule into a sequence string representation of the chemical molecule; masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph; generating, using the machine learning model, a concise representation comprising a hash vector representing the plurality of discontinuous substrings in the sequence string representation, wherein the machine learning model comprises a set of transformer structures; and training the set of transformer structures of the machine learning model to predict the hash vector. As a result, these illustrative embodiments provide a technical effect of training the set of transformer structures of the machine learning model to predict the hash vector.

In some embodiments, the sequence string representation comprises at least one of a SMILES representation and a SELFIES representation. As a result, these illustrative embodiments provide a technical effect of training the set of transformer structures of the machine learning model to predict the hash vector.

In some embodiments, masking comprises masking all tokens corresponding to atoms in the plurality of connected subgraphs in the molecular graph within a non-zero maximum distance r from an origin atom A. As a result, these illustrative embodiments provide a technical effect of training the set of transformer structures of the machine learning model to predict the hash vector.

In some embodiments, generating the concise representation comprising the hash vector representing the plurality of discontinuous substrings in the sequence string representation comprises iteratively hashing a concatenation of atoms and bonds within the non-zero maximum distance r from the origin atom A. As a result, these illustrative embodiments provide a technical effect of training the set of transformer structures of the machine learning model to predict the hash vector.

In some embodiments, the molecular graph comprises a 3-dimensional molecular graph. As a result, these illustrative embodiments provide a technical effect of training the set of transformer structures of the machine learning model to predict the hash vector.

Some embodiments further comprises, after coding, masking at least one individual token in the sequence string representation. As a result, these illustrative embodiments provide a technical effect of training the set of transformer structures of the machine learning model to predict the hash vector.

In some embodiments, training the set of transformer structures of the machine learning model comprises training to predict both the hash vector and the at least one individual token in the sequence string representation. As a result, these illustrative embodiments provide a technical effect of training the set of transformer structures of the machine learning model to predict the hash vector.

Some embodiments further include: masking, using the machine learning model, all tokens in the sequence string representation corresponding to another chemical substructure within the molecular graph of the chemical molecule using another plurality of discontinuous substrings in the sequence string representation; and generating, using the machine learning model, another concise representation comprising another hash vector representing the plurality of discontinuous substrings in the sequence string representation, wherein the machine learning model comprises a set of transformer structures, wherein training the set of transformer structures of the machine learning model comprises training the set of transformer structures of the machine learning model comprises to predict the another hash vector. As a result, these illustrative embodiments provide a technical effect of training the set of transformer structures of the machine learning model to predict the hash vector.

Some embodiments further include assessing, using the machine learning model, at least one drug property of a candidate chemical molecule based on at least one of absorption, distribution, metabolism, excretion, and toxicity. As a result, these illustrative embodiments provide a technical effect of training the set of transformer structures of the machine learning model to predict the hash vector.

Some embodiments further include, responsive to assessing, chemically synthesizing the chemical molecule. As a result, these illustrative embodiments provide a technical effect of training the set of transformer structures of the machine learning model to predict the hash vector.

Some embodiments further include, responsive to assessing, trialing the molecule comprising administering at least one sample of the chemical molecule to at least one specimen. As a result, these illustrative embodiments provide a technical effect of training the set of transformer structures of the machine learning model to predict the hash vector.

Embodiments include a computer system comprising: a processor set; a set of one or more computer readable storage media; program instructions, collectively stored in the set of one or more storage media, for causing the processor set to perform the following computer operations: encoding, using the processor set, a molecular graph of a chemical molecule into a sequence string representation of the chemical molecule; masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph; generating, using the machine learning model, a concise representation comprising a hash vector representing the plurality of discontinuous substrings in the sequence string representation, wherein the machine learning model comprises a set of transformer structures; and training the set of transformer structures of the machine learning model to predict the hash vector. As a result, these illustrative embodiments provide a technical effect of training the set of transformer structures of the machine learning model to predict the hash vector.

In some embodiments, the sequence string representation comprises at least one of a SMILES representation and a SELFIES representation. As a result, these illustrative embodiments provide a technical effect of training the set of transformer structures of the machine learning model to predict the hash vector.

In some embodiments, masking comprises masking all tokens corresponding to atoms in the plurality of connected subgraphs in the molecular graph within a non-zero maximum distance r from an origin atom A. As a result, these illustrative embodiments provide a technical effect of training the set of transformer structures of the machine learning model to predict the hash vector.

In some embodiments, generating the concise representation comprising the hash vector representing the plurality of discontinuous substrings in the sequence string representation comprises iteratively hashing a concatenation of atoms and bonds within the non-zero maximum distance r from the origin atom A. As a result, these illustrative embodiments provide a technical effect of training the set of transformer structures of the machine learning model to predict the hash vector.

In some embodiments, the molecular graph comprises a 3-dimensional molecular graph. As a result, these illustrative embodiments provide a technical effect of training the set of transformer structures of the machine learning model to predict the hash vector.

Embodiments include a computer program product comprising: a set of one or more computer-readable storage media; and program instructions, collectively stored in the set of one or more storage media, for causing a processor set to perform the following computer operations: encoding, using a set of processors, a molecular graph of a chemical molecule into a sequence string representation of the chemical molecule; masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph; generating, using the machine learning model, a concise representation comprising a hash vector representing the plurality of discontinuous substrings in the sequence string representation, wherein the machine learning model comprises a set of transformer structures; and training the set of transformer structures of the machine learning model to predict the hash vector. As a result, these illustrative embodiments provide a technical effect of training the set of transformer structures of the machine learning model to predict the hash vector.

In some embodiments, the sequence string representation comprises at least one of a SMILES representation and a SELFIES representation. As a result, these illustrative embodiments provide a technical effect of training the set of transformer structures of the machine learning model to predict the hash vector.

In some embodiments, masking comprises masking all tokens corresponding to atoms in the plurality of connected subgraphs in the molecular graph within a non-zero maximum distance r from an origin atom A. As a result, these illustrative embodiments provide a technical effect of training the set of transformer structures of the machine learning model to predict the hash vector.

In some embodiments, generating the concise representation comprising the hash vector representing the plurality of discontinuous substrings in the sequence string representation comprises iteratively hashing a concatenation of atoms and bonds within the non-zero maximum distance r from the origin atom A. As a result, these illustrative embodiments provide a technical effect of training the set of transformer structures of the machine learning model to predict the hash vector.

In some embodiments, the molecular graph comprises a 3-dimensional molecular graph. As a result, these illustrative embodiments provide a technical effect of training the set of transformer structures of the machine learning model to predict the hash vector.

Embodiments include a computer implemented method, comprising: encoding, using a set of processors, a molecular graph of a chemical molecule into a sequence string representation of the chemical molecule; masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph; generating, using the machine learning model, a concise representation comprising a hash vector representing the plurality of discontinuous substrings in the sequence string representation, wherein the machine learning model comprises a set of transformer structures; and training the set of transformer structures of the machine learning model to predict the hash vector, wherein the sequence string representation comprises at least one of a SMILES representation and a SELFIES representation, and wherein masking comprises masking all tokens corresponding to atoms in the plurality of connected subgraphs in the molecular graph within a non-zero maximum distance r from an origin atom A. As a result, these illustrative embodiments provide a technical effect of training the set of transformer structures of the machine learning model to predict the hash vector.

In some embodiments, generating the concise representation comprising the hash vector representing the plurality of discontinuous substrings in the sequence string representation comprises iteratively hashing a concatenation of atoms and bonds within the non-zero maximum distance r from the origin atom A. As a result, these illustrative embodiments provide a technical effect of training the set of transformer structures of the machine learning model to predict the hash vector.

Embodiments include a computer system comprising: a processor set; a set of one or more computer readable storage media; program instructions, collectively stored in the set of one or more storage media, for causing the processor set to perform the following computer operations: encoding, using the processor set, a molecular graph of a chemical molecule into a sequence string representation of the chemical molecule; masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph; generating, using the machine learning model, a concise representation comprising a hash vector representing the plurality of discontinuous substrings in the sequence string representation, wherein the machine learning model comprises a set of transformer structures; and training the set of transformer structures of the machine learning model to predict the hash vector, wherein the sequence string representation comprises at least one of a SMILES representation and a SELFIES representation, and wherein masking comprises masking all tokens corresponding to atoms in the plurality of connected subgraphs in the molecular graph within a non-zero maximum distance r from an origin atom A. As a result, these illustrative embodiments provide a technical effect of training the set of transformer structures of the machine learning model to predict the hash vector.

In some embodiments, generating the concise representation comprising the hash vector representing the plurality of discontinuous substrings in the sequence string representation comprises iteratively hashing a concatenation of atoms and bonds within the non-zero maximum distance r from the origin atom A. As a result, these illustrative embodiments provide a technical effect of training the set of transformer structures of the machine learning model to predict the hash vector.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner that at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

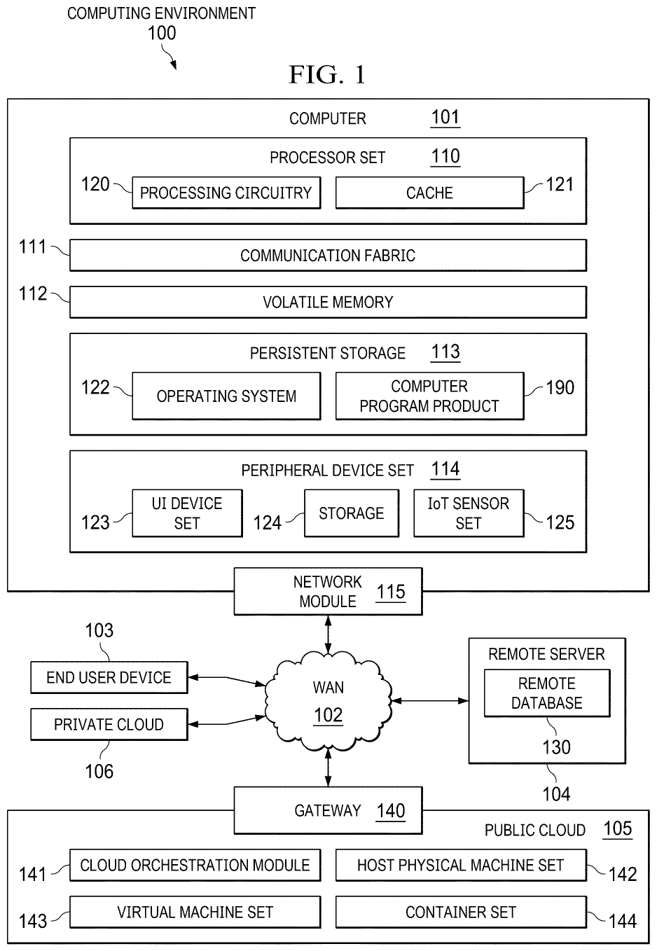

With reference now to the figures in particular with reference to FIG. 1, a block diagram of a computing environment is depicted in accordance with an illustrative embodiment. Computing environment 100 contains an example of an environment for the execution of at least some of the computer code involved in performing the inventive methods, such as predicting, using an artificial intelligence enabled system, an incremental degradation of a battery of a transportation vehicle, calculating an equivalent carbon footprint, calculating an equivalent carbon footprint tax, and assessing the tax against the vehicle. Embodiments of this disclosure can be embodied in computer program product 190. In addition to computer program product 190, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and computer program product 190, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile sequestering device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in computer program product 190 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in computer program product 190 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer, and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

In the illustrative examples, the hardware can take a form selected from at least one of a circuit system, an integrated circuit, an application specific integrated circuit (ASIC), a programmable logic device, or some other suitable type of hardware configured to perform a number of operations. With a programmable logic device, the device can be configured to perform the number of operations. The device can be reconfigured at a later time or can be permanently configured to perform the number of operations. Programmable logic devices include, for example, a programmable logic array, a programmable array logic, a field programmable logic array, a field programmable gate array, and other suitable hardware devices. Additionally, the processes can be implemented in organic components integrated with inorganic components and can be comprised entirely of organic components excluding a human being. For example, the processes can be implemented as circuits in organic semiconductors.

As used herein, “a number of” when used with reference to items, means one or more items. For example, “a number of parameters” is one or more parameters. As another example, “a number of operations” is one or more operations.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C, or item B and item C. Of course, any combination of these items can be present. In some illustrative examples, “at least one of” can be, for example, without limitation, two of item A; one of item B; and ten of item C; four of item B and seven of item C; or other suitable combinations.

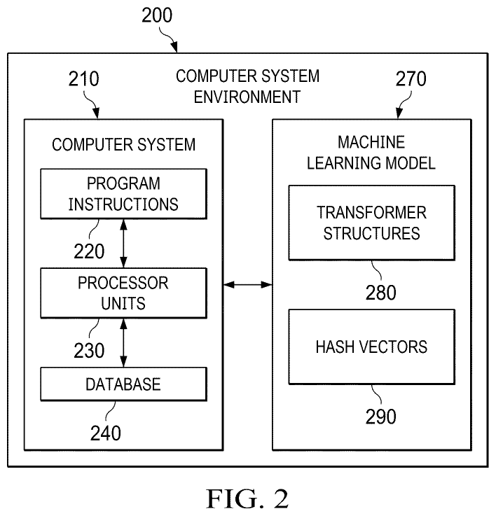

With reference now to FIG. 2, a block diagram of a computer system environment 200 is depicted in accordance with an illustrative embodiment. In this illustrative example, computer system environment 200 includes components that can be implemented in hardware such as the hardware shown in computing environment 100 in FIG. 1.

Computer system environment 200 includes computer system 210. Computer system 210 includes program instructions 220. Computer system 210 includes processor units 230. Computer system 210 includes database 240. Program instructions 220, processor units 230 and database 240 interact with one another.

The computer system environment 200 also includes machine learning model 270. Machine learning model 270 includes transformer structures 280. Machine learning model 270 includes hash vectors 290. Machine learning model 270 interacts with computer system 210.

Program instructions 220 and database 240 may be termed a machine learning model host. In particular, program instructions 220 and database 240 may be deployed with and/or implemented using computer program product 190 in FIG. 1.

Program instructions 220 and database 240 can be implemented in software, hardware, firmware or a combination thereof. When software is used, the operations performed by program instructions 220 and database 240 can be implemented using program instructions 220 configured to run on hardware, such as processor units 230. When firmware is used, the operations performed by program instructions 220 and database 240 can be implemented in program instructions and data and stored in persistent memory to run on a processor unit. When hardware is employed, the hardware can include circuits that operate to perform the operations in program instructions 220 and database 240.

Computer system 210 is a physical hardware system and includes one or more data processing systems. When more than one data processing system is present in computer system 210, those data processing systems are in communication with each other using a communications medium. The communications medium can be a network. The data processing systems can be selected from at least one of a computer, a server computer, a tablet computer, or some other suitable data processing system.

As depicted, computer system 210 includes processor units 230 that are capable of executing program instructions 220 implementing processes in the illustrative examples. In other words, program instructions 220 are computer readable program instructions.

As used herein, a processor unit in processor units 230 is a hardware device and is comprised of hardware circuits such as those on an integrated circuit that respond to and process instructions and program code that operate a computer. A processor unit can be implemented using processor set 110 in FIG. 1. When processor units 230 execute program instructions 220 for a process, processor units 230 can be one or more processor units that are in the same computer or in different computers. In other words, the process can be distributed between processor units on the same or different computers in computer system 210.

Further, the processor units 230 can be of the same type or different types of processor units. For example, the processor units 230 can be selected from at least one of a single core processor, a dual-core processor, a multi-processor core, a general-purpose central processing unit (CPU), a graphics processing unit (GPU), a digital signal processor (DSP), or some other type of processor unit.

Computer system 210 can be configured to perform at least one of the steps, operations, or actions described in the different illustrative examples using software, hardware, firmware or a combination thereof. As a result, computer system 210 operates as a special purpose computer system in which program instructions 220 and database 240 in computer system 210 enables a computer implemented method for drug discovery, comprising: encoding, using a set of processors, a molecular graph of a candidate molecule into a sequence representation of the candidate molecule; masking, using a pretrained machine learning model comprising transformer structures, at least one chemical substructure within the sequence representation with a plurality of tokens that define a first plurality of discontinuous substrings in the sequence representation; replacing, using the pretrained machine learning model comprising transformer structures, a plurality of tokens in the sequence representation corresponding to the at least one chemical substructure with a concise representation comprising a hash vector that uniquely identifies the at least one chemical substructure; and responsive to replacing the plurality of tokens, assessing, using the pretrained machine learning model, at least one drug property of the candidate molecule including absorption, distribution, metabolism, excretion, and toxicity based on the hash vector. In particular, program instructions 220 and database 240 transforms computer system 210 into a special purpose computer system as compared to currently available general computer systems that do not have program instructions 220 and database 240 because of the special purpose steps enabled by program instructions 220 and database 240.

In the illustrative example, the use of program instructions 220 and database 240 in computer system 210 integrates machine learning model 270 into a practical application such as, for example, assessing at least one drug property of a candidate molecule including absorption, distribution, metabolism, excretion, and toxicity. In other words, program instructions 220, processor units 230, and database 240 in computer system 210 integrate machine learning model 270 into practical technological application.

The illustration of the computer system 212 and computer system environment 200 in FIG. 2 is not meant to imply physical or architectural limitations to the manner in which an illustrative embodiment can be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

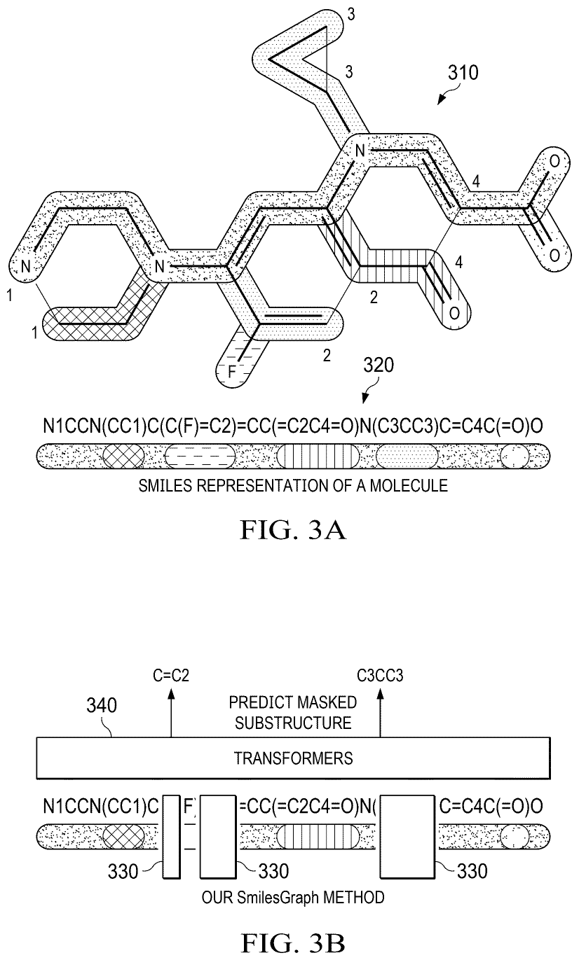

Referring to FIGS. 3A-3B, progression from structural formula to trained prediction of masked substructure via transformers is shown. FIG. 3A shows a structural formula 310 of a molecule. FIG. 3A also shows a SMILES representation 320 of the same molecule. FIG. 3B shows discontinuous masking 330 of the SMILES representation and predicted masked substructure from transformers 340.

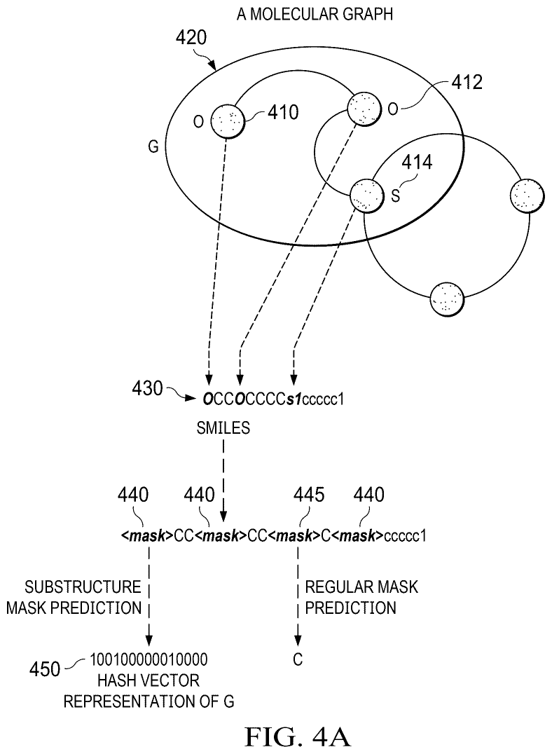

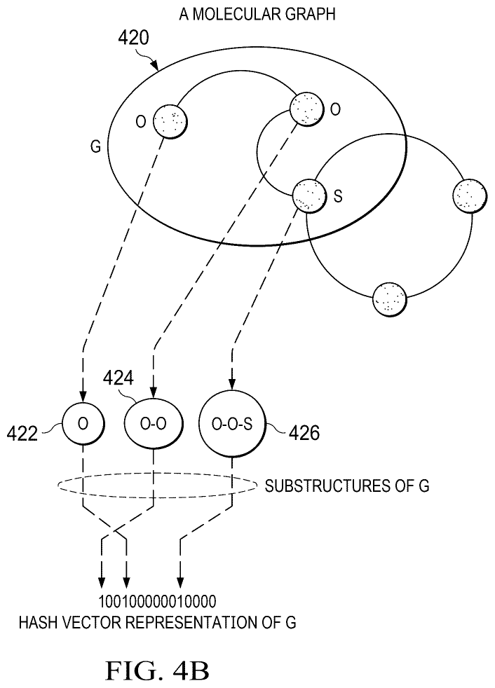

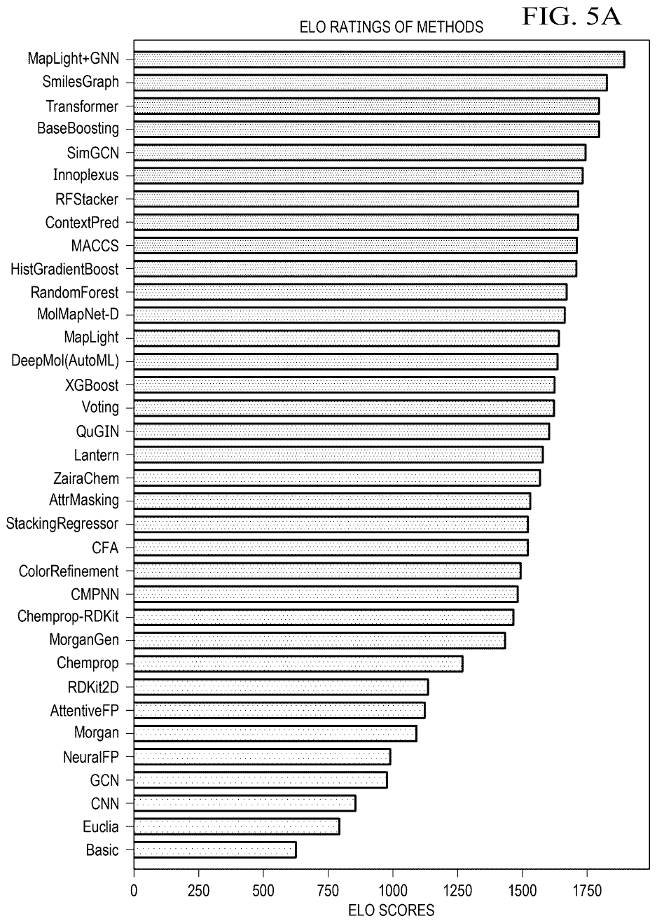

Referring to FIGS. 4A-4B, a transformation from a molecular graph to a SMILES representation to a hash vector representation is shown. A solution can include a pretrained model based on transformer structures called SMILES graphs. A model that learns to predict a concise representation of substructures can improve learning. In this method, the SMILES/SELFIES string is masked by replacing all tokens corresponding to a substructure with a concise representation based on random hashing. Transformers are then trained to predict this hash vector, allowing them to better capture and understand substructure rule information.

Referring to FIG. 4A, a random atom A 410 in the molecular graph is chosen. In this case, that atom is an oxygen atom. Start a breath first search from the chosen atom to find all neighboring atoms within a maximum distance that is a hyperparameter. This enables masking all tokens corresponding to atoms in a plurality of connected subgraphs in a molecular graph within a non-zero maximum distance r from an origin atom A. In this case, that maximum distance r is 2. The maximum distance can be termed r or R. Consequently, denote the graph in the neighborhood as G 420. G can alternatively be termed g. G defines a substructure. In FIG. 4A, atom A 410 was chosen (i.e. an oxygen atom) and its neighbor graph G 420 includes O atom 412 and S atom 414 where R (or r) equals 2.

SMILES representation 430 is then masked. In this case, the neighbor graph G receives 3 discontinuous masks 440. In general, embodiments can mask all atoms found in the neighbor graph G in the SMILES representation. Incidentally, another individual C atom also receives a mask 445 in this particular case.

Create a hash vector 450 representation of the neighborhood graph G by iteratively hashing the concatenation of the atoms and the bonds in the breadth first search. This hash vector is considered as the concise representation of the corresponding graph.

Training can be done by predicting the atoms 410, 412, 414 masked and transformed into the concise representation of the substructure(s). Since a substructure is represented as a hash vector, this approach avoids the enumeration of all possible substructures which could be prohibited large numbers. Embodiments of this disclosure can be termed Smilesgraph.

Referring to FIG. 4B, it can be appreciated that in this case neighbor graph G 420 (or g) contains further (sub)substructures 422, 424, 426. An exemplary correspondence between the individual (sub)substructures and components of the hash vector 450 is also shown in FIG. 4B with dashed line arrows.

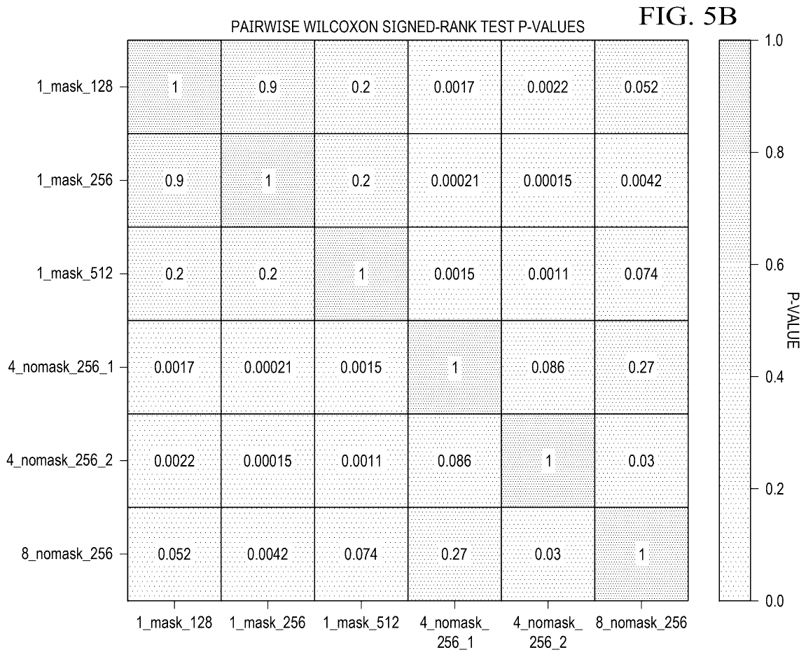

Referring to FIGS. 5A-5B, commercially advantageous results exhibited by embodiments of the present disclosure are shown. FIG. 5A shows that transformers trained with the substructure-aware masking strategy outperform traditional random masking on 22 ADMET benchmarks, leading to more accurate predictions of drug-related properties. These embodiments ranked second in Elo rating among 34 baseline methods and achieved comparatively superior results in four tasks, highlighting its competitive advantage in improving transformer performance for molecular representation learning. FIG. 5B shows p-values from the Wilcoxon signed-rank test comparing the SmilesGraph model across different settings: substructure count, masking, and hash vector size (e.g., 8 nomask 256 indicates a model with no masking, a hash size of 256, and 8 substructures). The results demonstrate that models using substructure asking significantly outperform those without. While the hash size does not have a statistically significant effect, smaller hash sizes tend to yield slightly better results. Additionally, the number of masked substructures has minimal impact in the nomask setting.

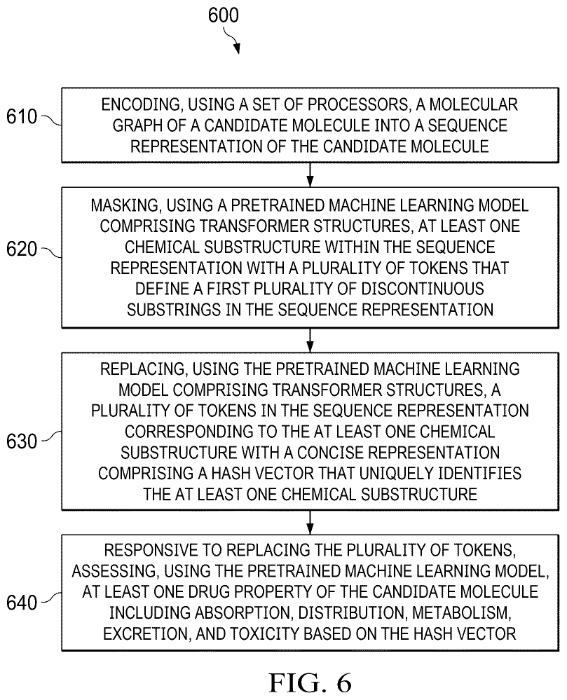

Referring to FIG. 6, a flowchart 600 of an embodiment of a computer implemented method for drug discovery is shown. Block 610 includes encoding, using a set of processors, a molecular graph of a candidate molecule into a sequence representation of the candidate molecule. Block 620 includes masking, using a pretrained machine learning model comprising transformer structures, at least one chemical substructure within the sequence representation with a plurality of tokens that define a first plurality of discontinuous substrings in the sequence representation. Block 630 includes replacing, using the pretrained machine learning model comprising transformer structures, a plurality of tokens in the sequence representation corresponding to the at least one chemical substructure with a concise representation comprising a hash vector that uniquely identifies the at least one chemical substructure. Block 640 includes responsive to replacing the plurality of tokens, assessing, using the pretrained machine learning model, at least one drug property of the candidate molecule including absorption, distribution, metabolism, excretion, and toxicity based on the hash vector.

The flowcharts and block diagrams in the different depicted embodiments illustrate the architecture, functionality, and operation of some possible implementations of apparatuses and methods in an illustrative embodiment. In this regard, each block in the flowcharts or block diagrams may represent at least one of a module, a segment, a function, or a portion of an operation or step. For example, one or more of the blocks can be implemented as program instructions, hardware, or a combination of the program instructions and hardware. When implemented in hardware, the hardware may, for example, take the form of integrated circuits that are manufactured or configured to perform one or more operations in the flowcharts or block diagrams. When implemented as a combination of program instructions and hardware, the implementation may take the form of firmware. Each block in the flowcharts or the block diagrams can be implemented using special purpose hardware systems that perform the different operations or combinations of special purpose hardware and program instructions run by the special purpose hardware.

In some alternative implementations of an illustrative embodiment, the function or functions noted in the blocks may occur out of the order noted in the figures. For example, in some cases, two blocks shown in succession can be performed substantially concurrently, or the blocks may sometimes be performed in the reverse order, depending upon the functionality involved. Also, other blocks can be added in addition to the illustrated blocks in a flowchart or block diagram.

In some implementations, a system and method enables transformers learning by order interaction between important substructures of molecules from smiles selfies strings. The substructures chosen can be selected based on their overall significant statistics in a database of molecular graphs.

An embodiment can include a method that chooses the tokens to mask the SMILES/SELFIES strings based on the substructures defined in the molecular graphs, a substructure is defined as a subgraph into deep molecular graph or a substructure with atoms that are close to each other in a 3D molecular graph, the transformers predict the tokens regarding the mass substructures to learn high order interaction between substructures of the molecules. An embodiment can include a method for creating a concise representation of substructure based on a random hash or pre-trained compacted representation vectors of substructures via constructive learning or auto encoder and a prediction objective that predict the hash vectors compacted representation vectors of the substructures.

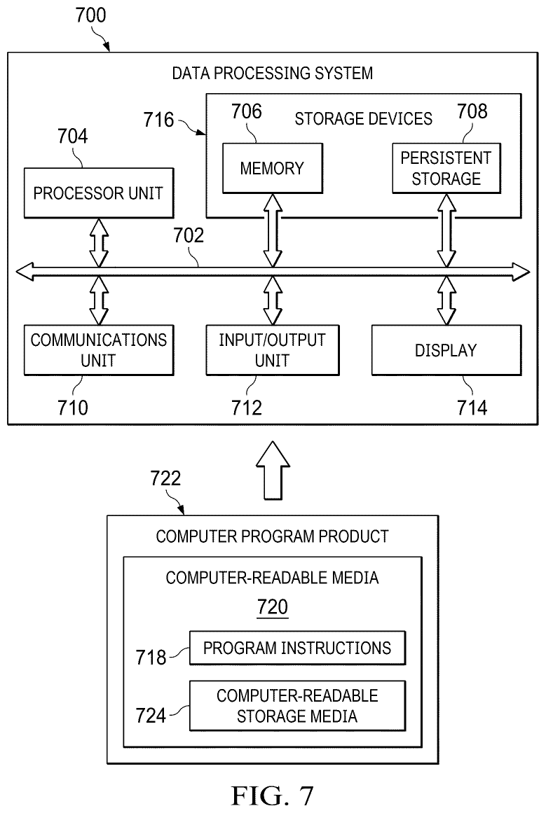

Turning now to FIG. 7, a block diagram of a data processing system is depicted in accordance with an illustrative embodiment. Data processing system 700 can be used to implement computers and computing devices in computing environment 100 in FIG. 1. Data processing system 700 can also be used to implement computer system environment 200 in FIG. 2. In this illustrative example, data processing system 700 includes communications framework 702, which provides communications between processor unit 704, memory 706, persistent storage 708, communications unit 710, input/output (I/O) unit 712, and display 714. In this example, communications framework 702 takes the form of a bus system.

Processor unit 704 serves to execute instructions for software that can be loaded into memory 706. Processor unit 704 includes one or more processors. For example, processor unit 704 can be selected from at least one of a multicore processor, a central processing unit (CPU), a graphics processing unit (GPU), a physics processing unit (PPU), a digital signal processor (DSP), a network processor, or some other suitable type of processor. Further, processor unit 704 can be implemented using one or more heterogeneous processor systems in which a main processor is present with secondary processors on a single chip. As another illustrative example, processor unit 704 can be a symmetric multi-processor system containing multiple processors of the same type on a single chip.

Memory 706 and persistent storage 708 are examples of storage devices 716. A storage device is any piece of hardware that is capable of storing information, such as, for example, without limitation, at least one of data, program instructions in functional form, or other suitable information either on a temporary basis, a permanent basis, or both on a temporary basis and a permanent basis. Storage devices 716 may also be referred to as computer readable storage devices in these illustrative examples. Memory 706, in these examples, can be, for example, a random-access memory or any other suitable volatile or non-volatile storage device. Persistent storage 708 may take various forms, depending on the particular implementation.

For example, persistent storage 708 may contain one or more components or devices. For example, persistent storage 708 can be a hard drive, a solid-state drive (SSD), a flash memory, a rewritable optical disk, a rewritable magnetic tape, or some combination of the above. The media used by persistent storage 708 also can be removable. For example, a removable hard drive can be used for persistent storage 708.

Communications unit 710, in these illustrative examples, provides for communications with other data processing systems or devices. In these illustrative examples, communications unit 710 is a network interface card.

Input/output unit 712 allows for input and output of data with other devices that can be connected to data processing system 700. For example, input/output unit 712 may provide a connection for user input through at least one of a keyboard, a mouse, or some other suitable input device. Further, input/output unit 712 may send output to a printer. Display 714 provides a mechanism to display information to a user.

Instructions for at least one of the operating system, applications, or programs can be located in storage devices 716, which are in communication with processor unit 704 through communications framework 702. The processes of the different embodiments can be performed by processor unit 704 using computer-implemented instructions, which may be located in a memory, such as memory 706.

These instructions are referred to as program instructions, computer usable program instructions, or computer readable program instructions that can be read and executed by a processor in processor unit 704. The program instructions in the different embodiments can be embodied on different physical or computer readable storage media, such as memory 706 or persistent storage 708.

Program instructions 718 are located in a functional form on computer-readable media 720 that is selectively removable and can be loaded onto or transferred to data processing system 700 for execution by processor unit 704. Program instructions 718 and computer-readable media 720 form computer program product 722 in these illustrative examples. In the illustrative example, computer-readable media 720 is computer-readable storage media 724.

Computer-readable storage media 724 is a physical or tangible storage device used to store program instructions 718 rather than a medium that propagates or transmits program instructions 718. Computer-readable storage media 724, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Alternatively, program instructions 718 can be transferred to data processing system 700 using a computer readable signal media. The computer readable signal media are signals and can be, for example, a propagated data signal containing program instructions 718. For example, the computer readable signal media can be at least one of an electromagnetic signal, an optical signal, or any other suitable type of signal. These signals can be transmitted over connections, such as wireless connections, optical fiber cable, coaxial cable, a wire, or any other suitable type of connection.

Further, as used herein, computer-readable media 720 can be singular or plural. For example, program instructions 718 can be located in computer-readable media 720 in the form of a single storage device or system. In another example, program instructions 718 can be located in computer-readable media 720 that is distributed in multiple data processing systems. In other words, some instructions in program instructions 718 can be located in one data processing system while other instructions in program instructions 718 can be located in one data processing system. For example, a portion of program instructions 718 can be located in computer-readable media 720 in a server computer while another portion of program instructions 718 can be located in computer-readable media 720 located in a set of client computers.

The different components illustrated for data processing system 700 are not meant to provide architectural limitations to the manner in which different embodiments can be implemented. In some illustrative examples, one or more of the components may be incorporated in or otherwise form a portion of, another component. For example, memory 706, or portions thereof, may be incorporated in processor unit 704 in some illustrative examples. The different illustrative embodiments can be implemented in a data processing system including components in addition to or in place of those illustrated for data processing system 700. Other components shown in FIG. 7 can be varied from the illustrative examples shown. The different embodiments can be implemented using any hardware device or system capable of running program instructions 718.

Thus, illustrative embodiments of the present disclosure provide a computer-implemented method, computer system, and computer program product for repositioning a sequestering device in a data center. The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer implemented method, comprising:
encoding, using a set of processors, a molecular graph of a chemical molecule into a sequence string representation of the chemical molecule;
masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph;
generating, using the machine learning model, a concise representation comprising a hash vector representing the plurality of discontinuous substrings in the sequence string representation, wherein the machine learning model comprises a set of transformer structures; and
training the set of transformer structures of the machine learning model to predict the hash vector.

2. The computer implemented method of claim 1, wherein the sequence string representation comprises at least one of a SMILES representation and a SELFIES representation.

3. The computer implemented method of claim 1, wherein masking comprises masking all tokens corresponding to atoms in the plurality of connected subgraphs in the molecular graph within a non-zero maximum distance r from an origin atom A.

4. The computer implemented method of claim 3, wherein generating the concise representation comprising the hash vector representing the plurality of discontinuous substrings in the sequence string representation comprises iteratively hashing a concatenation of atoms and bonds within the non-zero maximum distance r from the origin atom A.

5. The computer implemented method of claim 3, wherein the molecular graph comprises a 3-dimensional molecular graph.

6. The computer implemented method of claim 1, further comprising, after coding, masking at least one individual token in the sequence string representation.

7. The computer implemented method of claim 6, wherein training the set of transformer structures of the machine learning model comprises training to predict both the hash vector and the at least one individual token in the sequence string representation.

8. The computer implemented method of claim 1, further comprising:
masking, using the machine learning model, all tokens in the sequence string representation corresponding to another chemical substructure within the molecular graph of the chemical molecule using another plurality of discontinuous substrings in the sequence string representation; and
generating, using the machine learning model, another concise representation comprising another hash vector representing the plurality of discontinuous substrings in the sequence string representation, wherein the machine learning model comprises a set of transformer structures,
wherein training the set of transformer structures of the machine learning model comprises training the set of transformer structures of the machine learning model comprises to predict the another hash vector.

9. The computer implemented method of claim 1, further comprising assessing, using the machine learning model, at least one drug property of a candidate chemical molecule based on at least one of absorption, distribution, metabolism, excretion, and toxicity.

10. The computer implemented method of claim 8, further comprising, responsive to assessing, chemically synthesizing the chemical molecule.

11. The computer implemented method of claim 8, further comprising, responsive to assessing, trialing the molecule comprising administering at least one sample of the chemical molecule to at least one specimen.

12. A computer system comprising:
a processor set;
a set of one or more computer readable storage media;
program instructions, collectively stored in the set of one or more storage media, for causing the processor set to perform the following computer operations:
encoding, using the processor set, a molecular graph of a chemical molecule into a sequence string representation of the chemical molecule;
masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph;
generating, using the machine learning model, a concise representation comprising a hash vector representing the plurality of discontinuous substrings in the sequence string representation, wherein the machine learning model comprises a set of transformer structures; and
training the set of transformer structures of the machine learning model to predict the hash vector.

13. The computer system of claim 12, wherein the sequence string representation comprises at least one of a SMILES representation and a SELFIES representation.

14. The computer system of claim 12, wherein masking comprises masking all tokens corresponding to atoms in the plurality of connected subgraphs in the molecular graph within a non-zero maximum distance r from an origin atom A.

15. The computer system of claim 14, wherein generating the concise representation comprising the hash vector representing the plurality of discontinuous substrings in the sequence string representation comprises iteratively hashing a concatenation of atoms and bonds within the non-zero maximum distance r from the origin atom A.

16. The computer system of claim 14, wherein the molecular graph comprises a 3-dimensional molecular graph.

17. A computer program product comprising:
a set of one or more computer-readable storage media; and
program instructions, collectively stored in the set of one or more storage media, for causing a processor set to perform the following computer operations:
encoding, using a set of processors, a molecular graph of a chemical molecule into a sequence string representation of the chemical molecule;
masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph;
generating, using the machine learning model, a concise representation comprising a hash vector representing the plurality of discontinuous substrings in the sequence string representation, wherein the machine learning model comprises a set of transformer structures; and
training the set of transformer structures of the machine learning model to predict the hash vector.

18. The computer program product of claim 17, wherein the sequence string representation comprises at least one of a SMILES representation and a SELFIES representation.

19. The computer program product of claim 17, wherein masking comprises masking all tokens corresponding to atoms in the plurality of connected subgraphs in the molecular graph within a non-zero maximum distance r from an origin atom A.

20. The computer program product of claim 19, wherein generating the concise representation comprising the hash vector representing the plurality of discontinuous substrings in the sequence string representation comprises iteratively hashing a concatenation of atoms and bonds within the non-zero maximum distance r from the origin atom A.

21. The computer program product of claim 19, wherein the molecular graph comprises a 3-dimensional molecular graph.

22. A computer implemented method, comprising:
encoding, using a set of processors, a molecular graph of a chemical molecule into a sequence string representation of the chemical molecule;
masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph;
generating, using the machine learning model, a concise representation comprising a hash vector representing the plurality of discontinuous substrings in the sequence string representation, wherein the machine learning model comprises a set of transformer structures; and
training the set of transformer structures of the machine learning model to predict the hash vector,
wherein the sequence string representation comprises at least one of a SMILES representation and a SELFIES representation, and
wherein masking comprises masking all tokens corresponding to atoms in the plurality of connected subgraphs in the molecular graph within a non-zero maximum distance r from an origin atom A.

23. The computer implemented method of claim 22, wherein generating the concise representation comprising the hash vector representing the plurality of discontinuous substrings in the sequence string representation comprises iteratively hashing a concatenation of atoms and bonds within the non-zero maximum distance r from the origin atom A.

24. A computer system comprising:
a processor set;
a set of one or more computer readable storage media;
program instructions, collectively stored in the set of one or more storage media, for causing the processor set to perform the following computer operations:
encoding, using the processor set, a molecular graph of a chemical molecule into a sequence string representation of the chemical molecule;
masking, using a machine learning model, all tokens in the sequence string representation corresponding to a chemical substructure within the molecular graph of the chemical molecule using a plurality of discontinuous substrings in the sequence string representation, wherein the plurality of discontinuous substrings in the sequence string representation correspond to a plurality of connected subgraphs in the molecular graph;
generating, using the machine learning model, a concise representation comprising a hash vector representing the plurality of discontinuous substrings in the sequence string representation, wherein the machine learning model comprises a set of transformer structures; and
training the set of transformer structures of the machine learning model to predict the hash vector,
wherein the sequence string representation comprises at least one of a SMILES representation and a SELFIES representation, and
wherein masking comprises masking all tokens corresponding to atoms in the plurality of connected subgraphs in the molecular graph within a non-zero maximum distance r from an origin atom A.

25. The computer system of claim 24, wherein generating the concise representation comprising the hash vector representing the plurality of discontinuous substrings in the sequence string representation comprises iteratively hashing a concatenation of atoms and bonds within the non-zero maximum distance r from the origin atom A.
